(19) United States
(12) Patent Application Publication
(10) Pub. No.: US 2005/0288499 A1
(43) Pub. Date: Dec. 29, 2005

(54) Modified guanosines having flavouring activity

(75) Inventors: Carola Furiosi, Mozzo (IT); Andrea Zoia, Mozzo (IT); Claudio Ghizzoni, Mozzo (IT); Enrico Colombo, Mozzo (IT)

Correspondence Address: BIRCH STEWART KOLASCH & BIRCH, PO BOX 747, FALLS CHURCH, VA, 22040-0747, US

(73) Assignee: Kerry Group Services International Limited, Co. Kerry (IE)

(21) Appl. No.: 10/533,071

(22) PCT Filed: Oct. 27, 2003
(86) PCT No.: PCT/EP03/11913

§ 371 Date: Aug. 18, 2005

(30) Foreign Application Priority Data
Oct. 30, 2002 (IT) MI2002A002306

(51) Int. Cl. C07H 19/04

(52) U.S. Cl. 536/26.1

(57) ABSTRACT

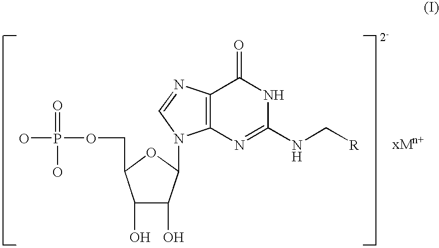

Guano sine-5′-monophosphate derivatives of general formula (I), wherein R, M, x and n have the meanings defined in the disclosure, are used as flavouring agents or flavour enhancers in alimentary products.

### FIELD OF THE INVENTION

The present invention relates to flavouring agents or flavour enhancers, in particular guanosine-5′-monophosphate (hereinafter also referred to as 5′-GMP) derivatives.

### SUMMARY OF THE INVENTION

The present invention relates to the compounds of general formula (I) 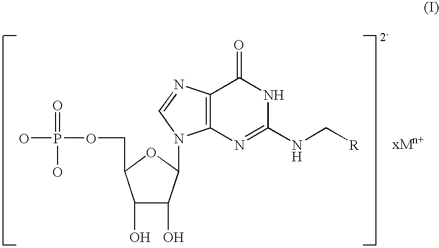
wherein R is:
   C₁-C₄ alkyl, optionally substituted with an S—R¹ group, in which R¹ is C₁-C₃ allyl;
   a phenyl, benzyl, thiophenyl or benzothiophenyl group, optionally substituted with one or more —NO₂, —CHO, —R¹ or —SR¹ groups, in which R¹ has the meaning defined above;
   M is hydrogen, an alkali or alkaline-earth metal;
   X is 1 when n is 2 and X is 2 when n is 1.

The compounds of formula (I) are particularly useful as excipients in the preparation of foods in which 5′-monophosphate ribonucleotides are used together with monosodium glutamate.

It is an object of the present invention the use of the compounds according to formula (I) as flavour enhancers and the use of a compound of formula (I) in admixture respectively with monosodium glutamate and/or 5′-monophosphate ribonucleotides.

### BACKGROUND OF THE INVENTION

The term “Flavour enhancer” indicates substances which do not necessarily have a flavour of their own but, when added to foods or flavourings, improve their organoleptic characteristics, increasing their savoury flavour and giving an impression of richness, meatiness, continuity and mouthfeel. This property is associated with a flavour known as the “fifth flavour”, called the “UMAMI flavour” by the Japanese.

Monosodium glutamate (hereinafter called “MSG”) is widely used as a flavour enhancer; however, in a certain category of consumers it is responsible for a series of side effects commonly known as “Chinese restaurant syndrome”.

These side effects can be partly reduced by associating 5′-monophosphate ribonucleotides, such as inosine 5′-monophosphate and guanosine 5′-monophosphate (hereinafter called IMP and GMP respectively), with MSG.

The use of modified ribonucleotides with superior activity to IMP and GMP would enable the quantity of MSG in foods and flavourings to be further reduced, thus reducing its side effects.

Yamazaki et al., Chem. Pharm. Bull., 1968, 16, 338-344 described the synthesis of 2-alkylthioinosine 5′-monophosphate and N²-methyl- and N²,N²-dimethylguanosine 5′-monophosphate, while K. Imai, Chem. Pharm. Bull., 1971, 19, 576-586 described the synthesis of 2-thio- and 2-arylinosine 5′-monophosphate.

In Communications, 80, 1025-1028, 1980, Keemal and Breeze described a general method for the synthesis of N²-alkyl nucleosides and the preparation of N²-methyl-guanosine. It has now been discovered that a class of N²-alkyl guanosines possess advantageous flavouring properties which enable the dose, and therefore the side effects, of MSG in foods and flavourings to be limited.

### DETAILED DISCLOSURE OF THE INVENTION

It is an object of the present invention the compounds of general formula (I) 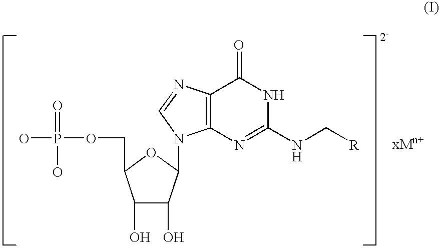
wherein R is:
   C₁-C₄ alkyl, optionally substituted with an S—R¹ group, in which R¹ is C₁-C₃ alkyl;
   a phenyl, benzyl, thiophenyl or benzothiophenyl group, optionally substituted with 1-5 substituents, preferably 1 to 3 substituents, which can be the same or different, selected from —NO₂, —CHO, —R¹ or —SR¹ groups, in which R¹ has the meaning defined above;
   M is hydrogen, an alkali or alkaline-earth metal;
   X is 1 when n is 2 and X is 2 when n is 1.

The C₁-C₄ alkyl group is selected from methyl, ethyl, propyl, isopropyl, n-butyl, sec-butyl, tert-butyl and preferably it is methyl or ethyl.

A first preferred group of compounds of formula (I) are those wherein R is a straight C₁-C₃ alkyl, optionally substituted with an S—R¹ group, in which R¹ is methyl.

A second preferred group of compounds of formula (I) are those wherein R is a phenyl, thiophenyl or benzothiophenyl group, optionally substituted with an —NO₂, methyl or —SR¹ group, in which R¹ has the meaning defined above.

A third preferred group of compounds of formula (I) are those wherein M is hydrogen, sodium, potassium, calcium, magnesium or barium and preferably sodium.

A fourth preferred group of compounds of formula (I) are those in which M is sodium, potassium, calcium, magnesium or barium and R is:
straight C₁-C₃ alkyl, optionally substituted with an —SR¹ group, in which R¹ corresponds to methyl;
a phenyl, thiophenyl or benzothiophenyl group, optionally substituted with an —NO₂, methyl or —SR¹ group, in which R¹ has the meaning defined above.

Furthermore, particularly preferred are the following compounds:
N²-3-(methylthio)-propyl guanosine-5′-monophosphate;
N²-(4-methylthiophenyl)-methyl guanosine-5′-monophosphate;
N²-(2-methylthiophenyl)-methyl guanosine-5′-monophosphate;
N²-(thiophen-2-yl)-methyl guanosine-5′-monophosphate;
N²-(thiophen-3-yl)-methyl guanosine-5′-monophosphate;
N²-(5-methylthiophen-2-yl)-methyl guanosine-5′-monophosphate;
N²-(3-methylthiophen-2-yl)-methyl guanosine-5′-monophosphate;
N²-(5-ethylthiophen-2-yl)-methyl guanosine-5′-monophosphate;
N²-(5-nitrothiophen-2-yl)-methyl guanosine-5′-monophosphate;
N²-(thianaphthen-3-yl)-methyl guanosine-5′-monophosphate
   and the corresponding sodium salts.

Most preferred compounds are N²-(3-methylthio)-propyl guanosine-55′-monophosphate and the corresponding sodium salt.

The compounds of general formula (I) have flavour enhancers activity equal to or higher than that of 5′-GMP. The introduction of the indicated substituents on the purine base causes a 2 to 20 times increase in activity, usually 2 to 10 times, compared with that of 5′-IMP, commonly used in the reference organoleptic evaluations.

A further object of the present invention is the use of the compounds of general formula (I), alone or in combination, as flavouring agents for increasing the flavoury savour typical of the products containing monosodium glutamate and corresponding derivatives in mammals and preferably in humans.

The compounds of formula (I) described above can therefore be used to prepare pasta, risottos, soups, dried and wet soups, snacks, sauces, salad dressings, ready-made red and white sauces, stock cubes, soup preparations, cooked dressed meats (ham, shoulder, reconstituted products, frankfurters, etc.) and uncooked dressed meats (salami, sausages, etc.), tinned meat, stuffed pasta and preserved vegetables, especially mushrooms.

The compounds of general formula (I) can be prepared with the process described in Kemal O., Communications, 1980, vol. 80, 1025-1028 (scheme), which allows to prepare purine ribonucleosides selectively modified at the 2-position.

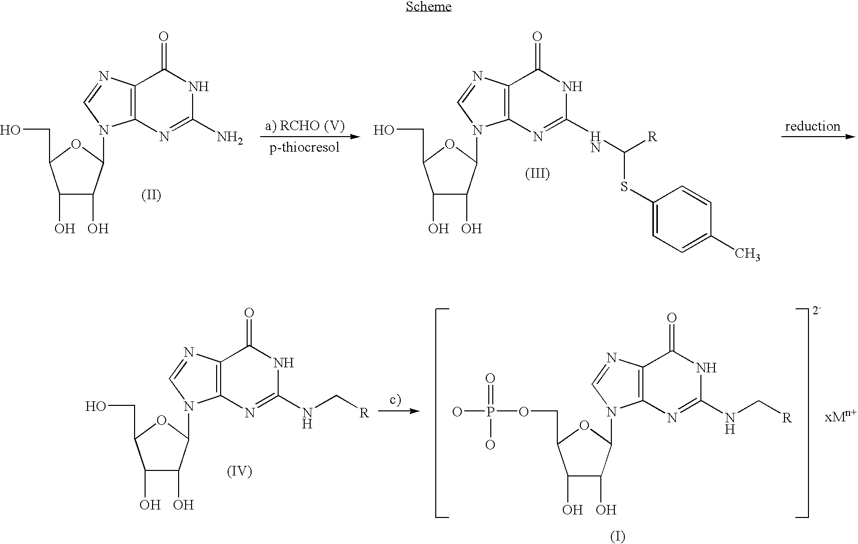

The process comprises reacting aldehydes R—CHO (V), in which R has the meanings defined above, and guanosine (II), in the presence of at least one mole of p-thiocresol, under acid catalytic conditions, preferably with acetic acid and preferably in aqueous medium, to yield the intermediate compounds of formula (III).

According to the present invention, preferred aldehydes are the following below indicated:
2-methylthiopropionaldehyde;
4-methylthiobenzaldehyde;
2-methylthiobenzaldehyde;
2-thiophenecarboxyaldehyde;
3-thiophenecarboxyaldehyde;
5-methyl-2-thiophenecarboxyaldehyde;
3-methyl-2-thiophenecarboxyaldehyde;
5-ethyl-2-thiophenecarboxyaldehyde;
5-nitro-2-thiophenecarboxyaldehyde;
thianaphthene-3-carboxaldehyde.

According to step a) of the Scheme, 2 to 40 mols of aldehyde of formula (V), 3 to 10 mols of p-thiocresol and an amount of acetic acid ranging from 2 to 15 mols are generally used.

The reaction is carried out preferably in aqueous, water-alcoholic or alcoholic solution, using a straight or branched C₁-C₄ alcohol, at a temperature usually ranging from 20 to 80° C. and is completed in a time from 2 to 24 hours.

The compounds of formula (III) are usually recovered from the reaction mixture and purified by crystallization, using for example aqueous ethanol.

In the subsequent step b), the compound of formula (III) is reduced to give the corresponding derivative of formula (IV). The reduction is preferably carried out with sodium borohydride in an anhydrous solvent and under similar conditions to those described in literature. 1 to 3 mols of reducing agent per mole of (IV) are preferably used, at a temperature from 40 to 100° C.; the reaction is usually completed in a time ranging from 2 to 4 hours.

Preferred solvents are 1,2-dimethoxyethane (glyme) and dimethylsulfoxide.

The phosphorylation reaction of N²-alkyl guanosine (IV), (step c), is carried out according to what described in K. Imai, J. Org. Chem., 1968, Vol. 34, No. 6, pp 1547-1550, using 1 to 4 mols of phosphorylating agent per mole of (IV) and at a temperature usually ranging from −10° C. to 0° C. Phosphoryl chloride (POCl₃) is preferably used in the presence of trimethylphosphate. The reaction is completed in a time comprising from 6 to 24 hours and the compound of formula (I) is isolated from the mixture, for example by crystallization with aqueous ethanol.

### EXPERIMENTAL SECTION

Organoleptic Properties

The organoleptic properties of the compounds of formula (I) were evaluated in sensory tests by convening a panel of 10 tasters trained to recognise the umami flavour.

The derivatives were evaluated in aqueous solution at a neutral pH in synergy with monosodium glutamate (MSG), using as standard an aqueous solution at a neutral pH containing sodium inosinate (5′-IMP) and MSG, in which the concentration of MSG remained fixed and the concentration of 5′-IMP was varied, until the point of subjective equality with the solution of the derivative to be evaluated was reached.

The compounds according to the invention can be used as flavour enhancers in savoury products, in which monosodium glutamate (MSG) and ribonucleotides (5′-IMP and 5′-GMP) are normally used, either in synergy with the salt (NaCl) or alone.

The flavour-enhancing activity of the compounds of formula (I) proved to be 2 to 20 times greater than that of 5′-IMP in synergy with MSG (reference solution).

The threshold values of the compounds of formula (I) in aqueous solution with a neutral pH varied within a range of 0.02 to 0.006 g/100 ml.

The dosage of the compounds of formula (I) in the finished product, falls within the range of 5 to 2500 ppm, when they are used as flavour enhancers.

For example, a dose which arrive to 2500 ppm of compounds of formula (I) is used in the specific case of stock cubes, whereas the dose is generally between 5 and 100 ppm of the finished product in the case of use as a generic flavouring.

The flavour quality of the compounds of formula (I) has a variety of connotations. The flavours include meat, vegetable, mushroom, spices, tomato, boiled potato, etc.

A number of applications in which the organoleptic properties of the new sulphurised ribonucleotide derivatives have been evaluated in finished products are described below.

Preparation 1: Stock Cube

The recipe (Table 1) uses a mixture of 5′-IMP and 5′-GMP, and the effect of replacing 5′-GMP with the new derivatives was evaluated.

TABLE 1

| INGREDIENTS | QUANTITY (g) |
|---|---|
| Salt | 50 |
| Monosodium glutamate (MSG) | 20 |
| Fats | 20 |
| Ribonucleotides (5′-GMP + 5′-IMP) | 0.5 |
| Meat extract | 2 |
| Vegetable extract - Yeast extract | 6.5 |
| Herbs and spices | 1 |
| | 100 |

The comparison was performed by taking the finished product according to the recipe set out in table 1 as reference.

The stock cube in the second formulation, containing guanosine sulphide derivatives, obtained scores 2 to 5 times higher than those of the reference formulation.

Other examples wherein the effect of the modified guanosine derivatives was evaluated are reported.

We chose commercial products which contain monosodium glutamate used as a flavour enhancer. We added a known quantity of compound of formula (I) (0.01 g) to these products, and performed the sensory evaluations by comparing the original product with the product to which the compound according to the invention was added.

Preparation 2: Sachet of Rice Preparation

TABLE 2

| INGREDIENTS | QUANTITY (g) |
|---|---|
| Parboiled rice | 84.32 |
| Dried artichoke | 7 |
| Fats | 3 |
| MSG | 2 |
| Salt | 1.5 |
| Powdered skimmed milk | 1 |
| White onion powder | 0.5 |
| Flavourings | 0.5 |
| Herbs and spices | 0.18 |
| | 100 |

Preparation 3: Meat-Based Filling for Fresh Pasta

TABLE 3

| INGREDIENTS | QUANTITY (g) |
|---|---|
| Pork and beef | 45 |
| Cheese | 20 |
| Breadcrumbs | 18 |
| Mortadella | 7 |
| Raw ham | 4 |
| Whey powder | 2 |
| Flavourings (*) | 1.5 |
| Salt | 1.5 |
| Parmesan cheese | 1 |
| | 100 |

(*) Monosodium glutamate in filling 0.4% (estimated)

In all cases, the products containing the compounds according to the invention obtained scores 2 to 5 times higher than those of the commercial product.

The example below illustrates the preparation of a compound of formula (I).

### N²-3′-methylthiopropyl guanosine-5′-monophosphate

### Step a): 2N-(p-tolylthio-3′-methylthiopropyl)-guanosine

In a 250 ml 2-necked round-bottom flask, equipped with condenser and thermometer, containing 45 ml of isopropanol, 3 g of guanosine (10.6 mmols), 4 g of p-thiocresol (32.2 mmols), 12 ml of methional (0.120 mols) and 6 ml of acetic acid are added under stirring and refluxed for 8-10 h.

After 2 h p-thiocresol (4 g) and 12 ml of methional are added again. The progress of the reaction is followed by TLC (eluent: ethyl acetate/ethanol/water=100:20:10).

The mixture is allowed to cool and evaporated under reduced pressure. The liquid residue is washed with ether to obtain a gummy, pale yellow solid (4.7 g, 89.6%).

### Step b): N²-3′-methylthiopropyl guanosine

In a 25 ml 3-necked round-bottom flask, equipped with condenser, thermometer and plug, containing a solution of compound prepared as in step a) (4.7 g, 9.5 mmols) in 8 ml of DMSO, 470 mg (12.4 mmols) of sodium borohydride are added, under stirring and the mixture is heated to 100° C. for 1.5 h, then cooled and neutralized with a 1 M solution of potassium dihydrogen phosphate. The resulting precipitate is filtered by suction and washed first with ice/water and then with sodium acetate.

3.5 g (94%) of a solid yellow product are obtained.

### Step c) N²-(3-methylthio)propyl guanosine-5′-monophosphate

2 ml of phosphoryl chloride are mixed with 13 ml of trimethyl phosphate and cooled to −10° C. in a 3-necked round-bottom flask with calcium chloride guard-tube and thermometer. The solution is added, in small portions and under stirring, with 3.5 g (9.4 mmol) of compound prepared in step b), keeping the temperature below −5° C. After approx. 30 minutes the solution becomes transparent and viscous.

The mixture is stirred for approx. 3 hours keeping the temperature at −5° C. The reaction is quenched by pouring the solution in 600 ml of ice/water, then pH is adjusted to 2 with 4 N sodium hydroxide. The solution is percolated on a column (diameter=3 cm) of animal activated charcoal (60 g), washed with distilled water to remove the salts and eluted with an ethanol/water/28% NH₄OH: 50/48/2 mixture.

The eluate is evaporated to dryness and the gummy residue is dissolved in 150 ml of water; pH is adjusted to 8.5 and to the solution is added under stirring 100 mg of barium acetate dissolved in some water.

The precipitate is centrifuged off and the resulting solution is added with 4 g of barium acetate in 30 ml of water. A further small portion of precipitate is obtained by addition of one volume of ethyl alcohol to the filtrate. The precipitate is isolated by centrifugation, then washed with ethanol, water and ether. The formed barium salt is suspended in 200 ml of water with 50 ml of Amberlite resin IR-120 (in Na+ form) and stirred until dissolution of the salt. The resin is filtered and the solution is reduced to 20 ml volume. Addition of ethyl alcohol yields an amorphous white precipitate (2.2 g, 51%).

1. Compounds of general formula (I) 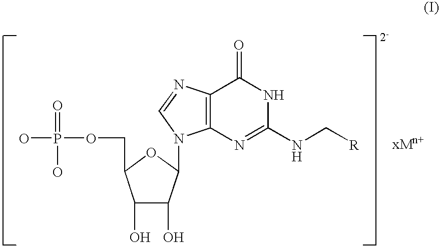
wherein, R is:
C₁-C₄ alkyl, optionally substituted with an S—R¹ group, in which R¹ is C₁-C₃ alkyl;
a phenyl, benzyl, thiophenyl or benzothiophenyl group, optionally substituted with 1-5 substituents, preferably 1 to 3 substituents, which can be the same or different, selected from —NO₂, —CHO, —R¹ or —SR¹ groups, in which R¹ has the meaning defined above;
M is hydrogen, an alkali or alkaline-earth metal;
X is 1 when n is 2 and x is 2 when n is 1 with the exclusion of the compounds of formula (i) wherein R is methyl or propyl.

2. Compounds as claimed in claim 1 wherein R is straight C₁-C₃ alkyl substituted with an S—R¹ group, in which R¹ is methyl.

3. Compounds as claimed in claim 1, wherein R is a phenyl, thiophenyl or benzothiophenyl group, optionally substituted with an —NO₂, methyl or —SR¹ group, in which R¹ is as defined in claim 1.

4. Compounds as claimed in claim 1, wherein M is hydrogen, sodium, potassium, calcium, magnesium or barium.

5. Compounds as claimed in claim 1, wherein M is hydrogen, sodium, potassium, calcium, magnesium or barium and R is:
straight C₁-C₃ alkyl substituted with a —SCH₃ group;
a phenyl, thiophenyl or benzothiophenyl group, optionally substituted with an —NO₂, methyl or —SCH₃ group.

6. Compounds as claimed in claim 1 in which M is hydrogen or sodium.

7. A compound as claimed in claim 1 which is selected from:
N²-(3-methylthio)-propyl guanosine-5′-monophosphate;
N²-(4-methylthiophenyl)-methyl guanosine-5′-monophosphate;
N²-(2-methylthiophenyl)-methyl guanosine-5′-monophosphate;
N²-thiophen-2-yl-methyl guanosine-5′-monophosphate;
N²-thiophen-3-yl-methyl guanosine-5′-monophosphate;
N²-(5-methylthiophen-2-yl)-methyl guanosine-5′-monophosphate;
N²-(3-methylthiophen-2-yl)-methyl guanosine-5′-monophosphate;
N²-(3-ethylthiophen-2-yl)-methyl guanosine-5′-monophosphate;
N²-(3-nitrothiophen-2-yl)-methyl guanosine-5′-monophosphate;
N²-(thianaphthen-3-yl)-methyl guanosine-5′-monophosphate
and the corresponding sodium salts.

8. A compound as claimed in claim 7 which is N²-(3-Methylthio)-propyl guanosine-5′-monophosphate and the corresponding sodium salt.

9. The use of the compounds as claimed in claim 1 as flavour enhancers in food preparations.

10. The use of the compounds as claimed in claim 1, alone or in combination with monosodium glutamate and/or with 5′-monophosphate nucleotides, as flavour enhancers in food preparations.

11. The use as claimed in claim 9 for the preparation of pasta, risottos, soups, dried and wet soups, snacks, sauces, salad dressings, ready-made red and white sauces, stock cubes, soup preparations, cooked and uncooked dressed meats, tinned meat, stuffed pasta and preserved vegetables.

12. Flavour enhancers comprising a compound of claim 1 in admixture with monosodium glutamate and/or with 5′-monophosphate nucleotides.

13. Food preparations containing the compositions of claim 12.

14. Compounds as claimed in claim 2, wherein M is hydrogen, sodium, potassium, calcium, magnesium or barium.

15. Compounds as claimed in claim 3, wherein M is hydrogen, sodium, potassium, calcium, magnesium or barium.

16. Compounds as claimed in claim 2 in which M is hydrogen or sodium.

17. Compounds as claimed in claim 3 in which M is hydrogen or sodium.

18. Compounds as claimed in claim 4 in which M is hydrogen or sodium.

19. Compounds as claimed in claim 5 in which M is hydrogen or sodium.
